(19) United States
(12) Patent Application Publication
(10) Pub. No.: US 2023/0104486 A1
(43) Pub. Date: Apr. 6, 2023

(54) LUNG IMAGING SYSTEM FOR TARGETED THERAPY

(71) Applicant: HILL-ROM SERVICES PTE. LTD., Singapore (SG)

(72) Inventors: Chang Sheng LEE, Punggol (SG); Yaolong LOU, Singapore (SG); Minghui LI, Singapore (SG); Ravinder S. DAHIYA, Glasgow (GB)

(21) Appl. No.: 17/960,538

(22) Filed: Oct. 5, 2022

Related U.S. Application Data
(60) Provisional application No. 63/252,250, filed on Oct. 5, 2021.

(51) Int. Cl. A61B 7/00; A61B 5/00

(52) U.S. Cl. CPC A61B 7/003; A61B 5/7203; A61B 5/742; A61B 5/6805; A61B 2562/0204; A61B 2562/046

(57) ABSTRACT

The present disclosure generally relates to a system and a method of assessing the lungs of a patient using acoustic mapping to provide an image of the lung function and modifying therapy applied based on the information gathered.

### CROSS-REFERENCE TO RELATED APPLICATIONS

This application claims priority under 35 U.S.C. § 119(e) to U.S. Provisional Application No. 63/252,250, filed Oct. 5, 2021, which is expressly incorporated by reference herein.

### BACKGROUND

The present disclosure relates to evaluating therapies used for treating lung diseases. More specifically, the present disclosure relates to an acoustic approach for mapping human lungs and for assessing lung function.

The human lung is susceptible to external particles through inhalation, particularly the bronchial tree, as it is directly connected with the ambient air. Hence, the airway wall of the bronchial tree is covered with airway surface liquid—specifically, the mucus layer, which traps foreign substances inhaled from the air, including germs and viruses. Mucus is carried from the respiratory tracts to the pharynx by mucociliary clearance (MCC), where it is ingested or coughed up. MCC is a natural defense mechanism that protects the lungs from the detrimental effects of inhaled substances. Chronic inflammation, cystic fibrosis, and some respiratory viral diseases cause mucous discharges to thicken. In such pathologies, therapeutical techniques can help the patients improve their breathing by eliminating the excess thick mucus that could not be done with MCC alone.

High-Frequency Chest Wall Oscillation (HFCWO) is a common airway clearance technique for patients with thick mucus and low MCC efficiency. HFCWO is defined as small oscillations of mechanical parts at relatively high frequencies, typically between 5 and 20 Hz, applied onto the patient's thorax for respiratory therapy. Traditional HFCWO devices such as the Vest 105 by Hillrom use an air-filled garment enclosing the patient chest to generate motion similar to MCC. The parameter setting and operation are purely empirical according to user experience. Modern HFCWO devices such as the Monarch, the AffloVest, and the RespIn 11 are equipped with multiple electromagnetic/pneumatic actuators that can be controlled individually, enabling a targeted therapy to the locations where infections or mucus buildups are focused (herein after referred to as a “nidus”) for an optimal therapeutic process. Therefore, knowledge of nidus positions in the airway is critical, and imaging has been identified as the potential approach to nidi localization.

The most direct way to access lung health condition is to visualize the lung by imaging. Lung health is usually provided by chest images from x-ray, computed tomography (CT), and magnetic resonance imaging (MRI) techniques. These techniques are suitable for visualizing the airways and lung pathology. However, the cumbersome and unwieldy equipment required to prepare these images require that the images be captured at the equipment and must not be impeded by foreign objects such as clothing, jewelry, or the like. Electrical impedance tomography (EIT) is an imaging technology that can be implemented to provide portability to patients, but still requires the removal of clothing and the like to apply electrodes on the patient's skin on their chest and back. Vibration response imaging (VRI) by acoustic signals is another technique that is portable to the patient, but also suffers the drawback of attaching multiple sensors to the patient's skin.

The use of high frequency chest wall oscillation (HFCWO) techniques are known to provide ongoing pulmonary therapy that may be varied in intensity, frequency, and location to provide therapy tailored to a particular patient. For example, the Monarch® Airway Clearance System available from Hill-Rom, Inc., Batesville, Ind., provides mobility with targeted kinetic energy and airflow to thin and mobilize secretions from the airways. The use of such a therapy can be optimized by using images of the lungs/airways to target the provision of therapy to those areas that are in most need of therapy. However, the use of the therapy must be interrupted to allow for images of the lungs/airways to be gathered to provide information for targeting the therapy.

Thus, there exists a need for an assessment tool that provides outcome measures that allows for frequent monitoring, allows for an equipment-to-patient approach, provides regional/localized information on lung function, and eliminates the subjective nature of assessment.

### SUMMARY

The present disclosure includes one or more of the features recited in the appended claims and/or the following features which, alone or in any combination, may comprise patentable subject matter.

According to a first aspect of the present disclosure, a method of developing an image of the function of a lung comprises positioning an array of microphones adjacent on a patient, the array of microphones being spaced from one another according to a predetermined pattern. The method also comprise collecting a signal from each of the microphones. The method further comprises processing the signal from each of the microphones to establish an intensity for each signal from each microphone at a first time. The method further comprises applying an interpolation routine to interpolate intensities at a predefined distribution between the microphones at the first time. The method still further comprises normalizing each of the actual and interpolated intensities according to a predetermined schedule. The method still yet further comprises generating an image of the lung at the first time by displaying the distribution of normalized intensities at the locations associated with each intensity.

In some embodiments, the method of the first aspect may include processing the signal from each of the microphones to establish an intensity for each signal from each microphone at a second time, applying an interpolation routine to interpolate intensities at a predefined distribution between the microphones at the second time, normalizing each of the actual and interpolated intensities according to a predetermined schedule, and generating an image of the lung at the second time by displaying the distribution of normalized intensities at the locations associated with each intensity.

In some embodiments, the method of the first aspect may include comparing the image at the second time to the image at the second time to determine a change in lung function in the interval between the first time and the second time.

In some embodiments, the method of the first aspect may include applying a noise filter to the normalized intensities prior to generating the image.

In some embodiments, the step of processing the signal includes applying a hybrid approach of signal processing that utilizes wavelet-based total variation and a Wiener filter to limit the signals to frequencies associated with lung function.

In some embodiments, the step of processing the signal includes applying a denoising filter to limit signals to frequencies associated with lung function.

In some embodiments the method of the first aspect may include the image at the first time is rendered graphically in gray scale.

According to a second aspect of the present disclosure, a garment wearable by and individual, the garment comprises an array of microphones and a computing device. The array of microphones is positioned on the garment in a predetermined pattern, the microphones positioned such when the garment is worn by a person, and the microphones are positioned to detect sounds emanating from the lungs and airways of the person. The computing device is positioned on the garment, the computing device including a processor and a memory device, the memory device including instructions that, when executed by the processor, cause the processor to collect a signal from each of the microphones, process the signal from each of the microphones to establish an intensity for each signal from each microphone at a first time, apply an interpolation routine to interpolate intensities at a predefined distribution between the microphones at the first time, normalize each of the actual and interpolated intensities according to a predetermined schedule, and generate an image of the lung at the first time by displaying the distribution of normalized intensities at the locations associated with each intensity.

In some embodiments of the second aspect, the memory device may include further instructions that, when executed by the processor, cause the processor to process the signal from each of the microphones to establish an intensity for each signal from each microphone at a second time, apply an interpolation routine to interpolate intensities at a predefined distribution between the microphones at the second time, normalize each of the actual and interpolated intensities according to a predetermined schedule, and generate an image of the lung at the second time by displaying the distribution of normalized intensities at the locations associated with each intensity

In some embodiments of the second aspect, the memory device may include further instructions that, when executed by the processor, cause the processor to process the signal by applying a hybrid approach of signal processing that utilizes wavelet-based total variation and a Wiener filter to limit the signals to frequencies associated with lung function.

In some embodiments of the second aspect, the memory device may include further instructions that, when executed by the processor, cause the processor to generate the images of the lung at the first and second time in grayscale, the grayscale based on the intensity of the signal at each location.

In some embodiments of the second aspect, the memory device may include instructions that, when executed by the processor, cause the processor to apply a noise filter to the normalized intensities prior to generating the images.

In some embodiments of the second aspect, the memory device may include further instructions that, when executed by the processor, cause the processor to apply a denoising filter to limit the signals to frequencies associated with lung function.

In some embodiments of the second aspect, the memory device may include further instructions that, when executed by the processor, cause the processor to generate the images of the lung at the first and second time in grayscale, the grayscale based on the intensity of the signal at each location.

In some embodiments of the second aspect, the memory device may include the garment further comprises a display supported on the garment, the display in communication with the computer device such that the images generated are displayed on the display on the garment.

In some embodiments of the second aspect, the memory device may include the microphones are embodied as micro-electro-mechanical system microphones.

In some embodiments of the second aspect, the memory device may include the microphones are operated passively.

According to a third aspect of the present disclosure, a diagnostic vest for a person comprises an array of acoustic sensors positioned on the vest in a predetermined pattern, a display device coupled to the vest, and a computer device operable to process signals from the array of sensors and generate an image on the display, the image indicative of the lung function of person wearing the vest.

In some embodiments of the third aspect, the computer device synchronizes the displayed image with the breathing rate of the person wearing the vest.

In some embodiments of the third aspect, the computer device processes the signals by applying a denoising filter operable to filter acoustic signals in frequencies not associated with lung function.

In some embodiments of the third aspect, the computer device interpolates between the acoustic sensors to establish the image displayed.

In some embodiments of the third aspect, the image is displayed in grayscale.

In some embodiments of the third aspect, the acoustic sensors operate passively.

In a fourth aspect of the present disclosure a method of assessing lungs of a patient is disclosed. The method comprises capturing a first acoustic signal from the lungs of the patient at a first time, processing the first acoustic signal, generating a first output from the first acoustic signal, capturing a second acoustic signal from the lungs of the patient at a second time, processing the second acoustic signal, and generating a second output from the second acoustic signal. The first output and the second output provide information about any pathological abnormalities found in the lungs of the patient.

In some embodiments of the fourth aspect, the at least one sensor may be used in capturing the first and the second acoustic signals. In some embodiments, the system processes the first and second acoustic signals to synchronize the first and second acoustic signals by a breathing rate of the patient. In some embodiments, the at least one sensor is a multi-microphone sensor array. In some embodiments, the at least one sensor includes a microphone and a stethoscope. In some embodiments, the lungs of the patient includes a left lung and a right lung, and the at least one sensor includes at least a first sensor on the left lung, and at least a second sensor on the right lung. In some embodiments, the at least one sensor includes at least a first sensor on an anterior side of the patient, and at least a second sensor on a posterior side of the patient.

In some embodiments of the fourth aspect, capturing the first acoustic signal includes generating a first intensity signal of the captured first acoustic signal and capturing the second acoustic signal includes generating a second intensity signal of the captured second acoustic signal. In some embodiments, capturing the first acoustic signal further includes capturing the first intensity signal by a multichannel analog-to-digital converter, and capturing the second acoustic signal further includes capturing the second intensity signal by a multichannel analog-to-digital converter. In some embodiments, the multichannel analog-to-digital converter has a minimum 8-bit level and a sampling rate of about 16 KHz. In other embodiments, a lower or higher sampling rate may be used.

In some embodiments of the fourth aspect, the first time is a first time interval, and wherein the second time is a second time interval. In some embodiments, processing the first acoustic signal includes applying a denoising filter to the first acoustic signal, and processing the second acoustic signal includes applying a denoising filter to the second acoustic signal.

In some embodiments of the fourth aspect, processing the first acoustic signal includes synchronizing the first acoustic signal and to the patient's breathing at the first time, and processing the second acoustic signal includes synchronizing the second acoustic signal to the patient's breathing at the second time.

In some embodiments of the fourth aspect, processing the first acoustic signal includes converting the first acoustic signal to at least one first image, and processing the second acoustic signal includes converting the second acoustic signal to at least one second image.

In some embodiments of the fourth aspect, generating the first output includes generating at least one first image, and generating the second output includes generating at least one second image. In some embodiments, the at least one first image is a 2D dynamic graphical representation of the first acoustic signal, and wherein the at least one second image is a 2D dynamic graphical representation of the second acoustic signal.

In some embodiments of the fourth aspect, generating the first output comprises generating at least one first regional intensity values, and wherein generating the second output comprises generating at least one second regional intensity values.

In fifth aspect, the present disclosure relates to a system for assessing the lungs of a patient. The system includes at least one sensor, a signal processor, and an image processor. The least one sensor captures a first acoustic signal from the lungs of a patient at a first position at a first time and a second acoustic signal from the lungs of the patient at a second position at a second time. The signal processor processes the first acoustic signal to generate a first processed signal, and processes the second acoustic signal captured to generate a second processed signal and the signal processor further processes the first and second acoustic signals to synchronize the first and second acoustic signals by the breathing rate of the patient. The image processor converts the first processed signal to a first output and the second processed signal to a second output. The first output and second output provides information about pathological abnormalities found in the lungs of the patient.

In some embodiments of the fifth aspect, the at least one sensor is attached to clothes worn by the patient. In some embodiments, the at least one sensor is a multi-microphone sensor array. In some embodiments, the at least one sensor includes a microphone and a stethoscope.

In some embodiments of the fifth aspect, the lungs of a patient includes a left lung and a right lung, and the at least one sensor includes at least a first sensor on the left lung and at least a second sensor on the right lung.

In some embodiments of the fifth aspect, the at least one sensor includes at least a first sensor on an anterior side of the patient and at least a second sensor on a posterior side of the patient.

In some embodiments of the fifth aspect, the at least one sensor generates a first intensity signal of the first acoustic signal and a second intensity signal of the second acoustic signal. In some embodiments, the at least one sensor captures the first intensity signal and the second intensity signal by a multichannel analog-to-digital converter. In some embodiments, the multichannel analog-to-digital converter has a minimum 8-bit level and a sampling rate of about 16 KHz. In other embodiments, a lower or higher sampling rate may be used.

In some embodiments of the fifth aspect, the first time is a first time interval, and the second time is a second time interval. In some embodiments of the current system, the signal processor applies a denoising filter to the first acoustic signal and the second acoustic signal. In some embodiments of the current system, the signal processor uses an acoustic intensity algorithm. In some embodiments of the current system, the signal processor synchronizes the first acoustic signal to the patient's breathing at the first time and the second acoustic signal to the patient's breathing at the second time.

In some embodiments of the fifth aspect, the image processor converts the first processed signal to at least one first image and the second processed signal to at least one second image. In some embodiments, the at least one first image is a 2D dynamic graphical representation of the first acoustic signal, and wherein the at least one second image is a 2D dynamic graphical representation of the second acoustic signal.

In some embodiments of the fifth aspect, the image processor converts the first processed signal to at least one first regional intensity value and the second processed signal to at least one second regional intensity value.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a diagrammatic representation of a patient wearing a vest garment of the present disclosure, the garment including a number of sensors positioned on the garment and operable to detect sounds from the patient's chest;

FIG. 2 is an illustration of sensors attached to a garment of FIG. 1, where the sensors may capture acoustic signals from the patient's lungs;

FIG. 3 is a flow diagram showing the functional relationship of the capture and analysis of acoustic signals including of a sensing unit, a signal processing unit, and an image processing unit used for converting an acoustic signal captured from a patient's lung to an image representing function of the lung;

FIG. 4 is an example of a computing device with a signal processing unit and an image processing unit used to process and convert an acoustic signal captured from a patient's lung to an output representing function of the lung;

FIG. 5A-5B is a flowchart illustrating the various steps in the process of converting an acoustic signal captured from a patient's lung to an output representing function of the lung;

FIG. 6 is an example of a dynamic grayscale acoustic image representing the acoustic signals from a patient's lungs over a given time interval;

FIG. 7 is a diagrammatic representation of an embodiment of a hybrid denoising model for providing a denoised signal representative of lung function;

FIG. 8 is graph showing the root-mean-squared error (RMSE) performance for various denoised filters of lung sound signals captured from a healthy lung;

FIG. 9 is a diagrammatic model of an airway tree of bifurcating segments of human respiratory system;

FIG. 10 is a diagrammatic line model of the enlarged segment of FIG. 9 and the equivalent circuit of the line model;

FIG. 11 is a model of respiratory airways of a human respiratory system by equivalent circuit with lumped parameters;

FIG. 12 is a standard branch of the model of FIG. 11;

FIG. 13 is table relating the incidence matrix, branch admittance vector, and branch voltage source vector of a model of a human respiratory system;

FIG. 14 is a matrix of an airway geometry with multiple sensing areas, the location of acoustic sensors being represented by the circles at the intersections of the horizontal and vertical lines;

FIG. 15 is a reference acoustic image generated from a lung sound signal;

FIG. 16 is a model of the reference signal generated from an airway pressure signal;

FIG. 17 is a diagrammatic representation of the location of sensors on the opposing lobes of a lung with the sensors being overlapped by 75% on the left side and 0% on the right side;

FIG. 18 is a chart illustrating the relation size of nidus detected to the sensing sensitivity overlap for each of a number of different sized sensors; and

FIG. 19 is a chart illustrating the number of sensors required to identify the length of an obstructed airway based on sensor size.

### DETAILED DESCRIPTION

The current disclosure describes a lung imaging system and a method for implementing a lung imaging system. The current disclosure also describes a signal processing approach for differentiating the adventitious lung sounds, such as crackle and wheeze, from healthy lung sounds. The lung imaging system utilizes information obtained from a respiratory sound signal (acoustic signal) and provides an assessment of a patient's lung function. As will be described below, acoustic signals from a patient at two different time points may be used to assess the lung function of the patient. The two different time points may be before and after the use of a therapy to treat the patient's lungs. Acoustic signals from a patient at the two different time points may be used to assess the therapy used to treat the patient between the two different time points. An acoustic intensity mapping tool may utilize information captured from an acoustic signal of the patient to assess the lung function of the patient.

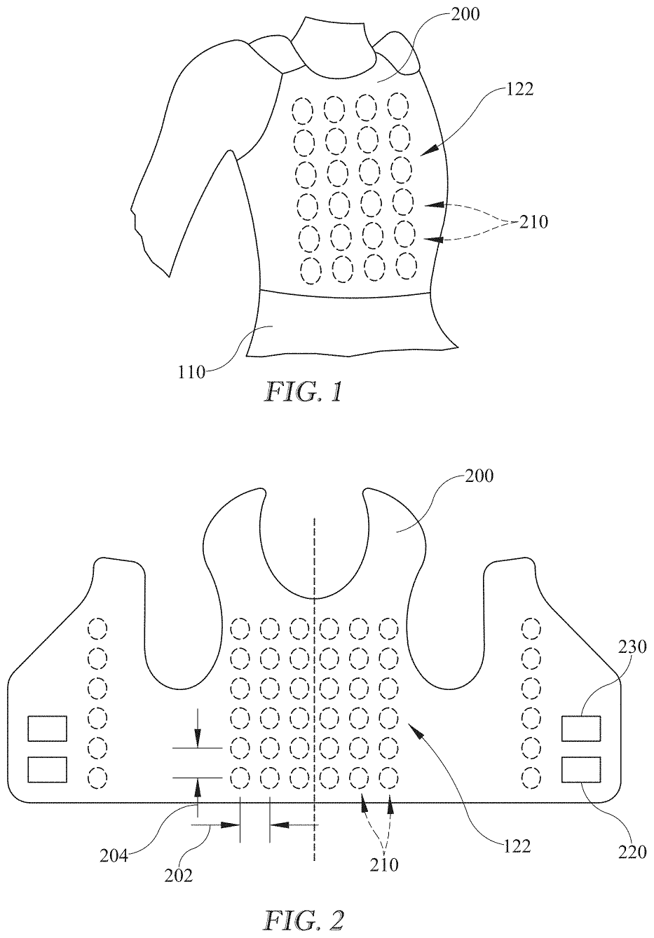

According to the present disclosure and as shown in FIGS. 1 and 2, one or more sensors 210 may be attached to a vest 200 to capture and process acoustic signals on both the left- and right-lung region of a patient 110. Such a mapping strategy may provide information on the respiratory health of both the left and right lungs. In other embodiments, the sensors 210 may be attached to any other piece of clothing or patient wearable device. In the disclosed embodiment, the sensors 210 are positioned at a distance designated as 202 in a first direction and designated as 204 in a second direction orthogonal to the first direction. The distances 202, 204 of the present disclosure are both about 50 mm from each other sensor 210. In other embodiments, the sensors 210 may be at a distance larger than 50 mm from each other in either direction or both directions. In some embodiments, the sensors 210 may be at a distance smaller than 50 mm from each other in either direction or both directions. Depending on the application, more or fewer sensors 210 may be included and the spacing may be varied depending on particular indications to change the sensitivity in particular regions of the lung, thereby reducing the interpolation needed to develop the images as will be described below.

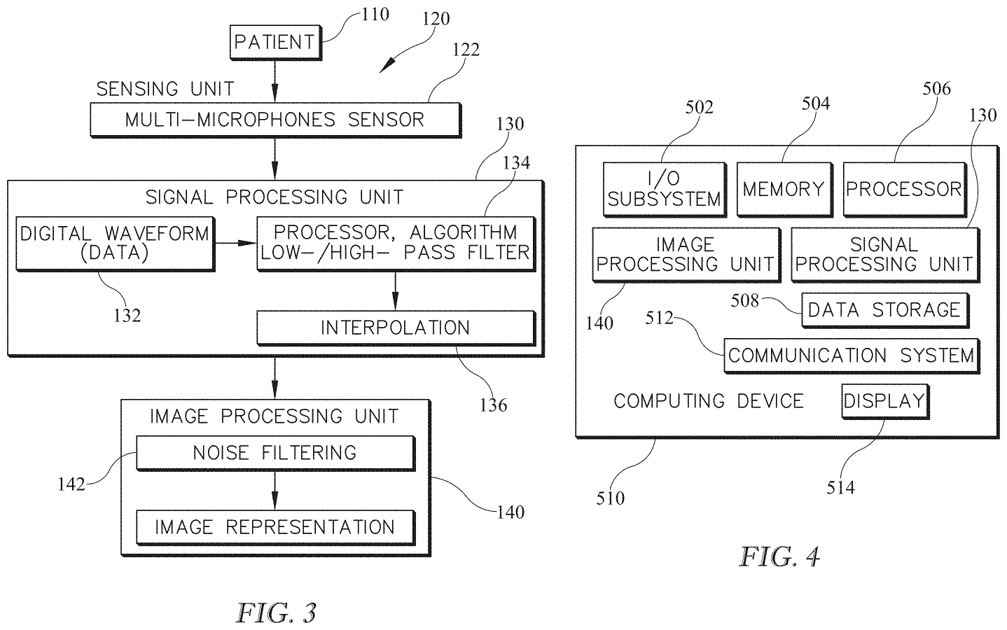

As shown diagrammatically in FIG. 3, a signal from the lungs of a patient 110 is captured by a sensing unit 120. The signal is then processed by a signal processing unit 130 to obtain an output in the form of one or more images. The images, as described further below, are processed by an image processing unit 140. Thus, an acoustic signal is converted to an image representing the lung function of a patient. In this context, the “image” is a digital construction, which may be visualized graphically, based on the intensity of sounds emanating from the lungs of the patient 110.

The information from the acoustic signal of the lungs of the patient is captured by the sensing unit 120 using a microprocessor and with the sensors 210 embodied as high signal-to-noise ratio (SNR) Micro-Electro-Mechanical System (MEMs) microphones arranged in an array 122. Microphone arrays are known for beamforming and are an established technology for sound source localization. The beamforming microphone array 122 gives information on the location of a sound source emitting from the lungs and aid in the diagnosis of lung pathological abnormalities. The present disclosure uses MEMs microphones to capture the information from a respiratory signal because MEMs microphones are small in size, have low power consumption, and respond to a change in pressure caused by respiratory sound waves. In one embodiment, the signals are captured by a multichannel analog-to-digital converter, with a minimum 8-bit level and a sampling rate of 16 KHz. In other embodiments, a lower or higher sampling rate may be used. As described in further detail below, the signal captured by the sensing unit 120 is processed and the lung function assessment is output in one or more images and regional intensity values.

The disclosed approach of using the MEMs microphone array 122 enables an equipment-to-patient approach, thereby minimizing the risk of transporting patients to traditional imaging equipment and minimizing the lack of direct access in the clinical setting. The MEMs microphone array 122 does not expose the patient to radiation of other imaging techniques, preventing any additional negative effect on the health of the patient 110.

In other embodiments, the MEMs microphone array 122 may be attached to any clothing on the patient. In other embodiments, the MEMs microphone array 122 may be attached directly to the body of the patient. In some embodiments, a microphone and a digital stethoscope may be used as a sensor 210. The MEMs microphone array 122 may be attached to the patient's posterior, and the microphone may generate an intensity signal captured from the patient's lung.

As shown in FIG. 2, individual sensors 210 may be positioned (modeled by position vector x) and configured for attachment on a planar region on a patient's posterior. In other embodiments, N sensors may be positioned (modeled by position vectors x) and configured for attachment on a planar region on the patient's posterior. The sensors 210 are each fixed at a position (i=1:N) in the planar region and generate a signal. The sensors 210 may be denoted by P(xᵢ, t), where xᵢ is the position of a particular sensor 210.

Referring back to FIG. 3, the captured signals go through an acoustic intensity algorithm for signal processing in a signal processing unit 130. Referring to FIG. 3, the data in the form of a digital waveform 132 is processed by a denoising filter 134 that allows only the desired frequency range of respiratory sounds to be captured. The average acoustic intensity may be computed over a given time interval from t₁ to t₂ at each sensor position xᵢ as described below.

\tilde{P}(xᵢ, t₁, t₂)=Σₜ_{₁}^{t}^{₂}P²(xᵢ, t) (1)

The acoustic intensity signals may then be converted to one or more images by the interpolation method 136 discussed below. The acoustic intensity at any location x is estimated using the interpolation method. A two dimensional data set to be interpolated is defined by the intensity function \tilde{P}(xᵢ, t₁, t₂), where the signal P is a finite set of a pixels. The interpolation method using equation (2)-(3) below is performed on \tilde{P}(xᵢ, t₁, t₂) at position x

[symbol]

(x, t₁, t₂)=Σᵢ₌₁^{N}\tilde{P}(xᵢ, t₁, t₂)g(x, xᵢ) (2)

where, g(x, xᵢ) is an interpolating kernel function.

g(x, xᵢ)=Σᵢ₌₀^{N}P(xⁱxᵢ^{j}) (3)

where, xᵢ=(xᵢ¹, xᵢ²) is the position of the sensor.

Since the positions xᵢ of the sensors 210 are discrete, a two-dimensional interpolation is exercised to develop the values at intermediate locations where sensors 210 are not present. The highest, lowest, and in-between values of the actual and interpolated signal values are set as black, white, and gray at each pixel, respectively. Thus, each signal recording is normalized to the black, white, gray values. In the illustrative embodiment, shown in FIG. 3, the acoustic signals captured by the sensors 210 are then processed by a controller 220 on the vest 200.

The one or more images obtained in 136 is processed in the image processing unit 140 with a noise filtering algorithm 142 before a two-dimensional dynamic graphical representation 144 of the intensity signals may be obtained. The output after signal processing maybe displayed on the vest 200 at an output display 230 shown in FIG. 2. The output at display 230 is displayed as the one or more images obtained from the acoustic intensity signal. In other embodiments, the output may be a different rendering of the acoustic intensity signal such as the hybrid approach of signal processing that utilizes wavelet-based total variation and a Wiener filter described below.

In this embodiment, the lung sound signal is modulated with amplitude and frequency modulators,

xₐ(t)=xₛ(t)mₐ(t)m_{f}(t), (4)

where xₐ(t) is the airflow hitting on the airway, xₛ(t) is the airflow; the amplitude and frequency modulation functions are denoted as mₐ(t) and m_{f}(t), respectively.

The modulated airflow is accompanied by noises when it penetrates the airway wall,

x_{f}(t)=xₐ(t)+vₐ(t), (5)

where x_{f}(t) is the airflow with accompanying noises after the airflow hits on the airway, and vₐ(t) is the accompanying noise when xₐ(t) hits the airway.

Other noise, including from the sensor 210 is also transferred according to:

x(t)=x_{f}(t)+v_{f}(t), (6)

where x(t) is the airflow that is transmitted to the chest wall or the modulated signal with noises, and v_{f}(t) is the noise transferred from the sensor 210.

There may also be ambient noise produced in the environment and other factors such as speech and cough during the lung sound recording which follows

y(t)=x(t)+vₑ(t), (7)

where y(t) is the airflow that is captured by the sensor 210 with noise, and vₑ(t) is the noise caused by ambient. Substituting (4)-(6) into (7), the lung sound containing noise follows:

y(t)=xₛ(t)mₐ(t)m_{f}(t)+vₐ(t)+v_{f}(t)+vₑ(t) (8)

A reasonable assumption is that the noises are zero-mean process having a probability density distribution that can be defined with mean and variance, uncorrelated with the transmitted lung sound x(t), with varying power levels as explained in D. Singh, B. K. Singh, and A. K. Behera, “Comparative analysis of Lung sound denoising technique,” in 2020 First International Conference on Power, Control and Computing Technologies (ICPC2T), 2020, pp. 406-410; Y. Ding and I. W. Selesnick, “Artifact-Free Wavelet Denoising: Non-convex Sparse Regularization, Convex Optimization,” IEEE Signal Processing Letters, vol. 22, no. 9, pp. 1364-1368, 2015; J. G. Gallaire and A. M. Sayeed, “Wavelet-based empirical Wiener filtering,” in Proceedings of the IEEE-SP International Symposium on Time-Frequency and Time-Scale Analysis (Cat. No. 98TH8380), 1998, pp. 641-644; and C. Hyeokho and R. Baraniuk, “Analysis of wavelet-domain Wiener filters,” in Proceedings of the IEEE-SP International Symposium on Time-Frequency and Time-Scale Analysis (Cat. No. 98TH8380), 1998, pp. 613-616, each of which incorporated herein by reference.

Based on the above noted assumption, the noises are modeled as white Gaussian noise (WGN) with vₐ(t), v_{f}(t), and vₑ(t) being combined. Therefore, (8) can be simplified to (9) similar to a linear system, where y(t) is the received lung sound signal (output) containing WGN (error) v(t) and the desired lung sound signal (input) xₐ(t) as in (4),

y(t)=xₐ(t)+v(t) (⁹)

From (9), the desired signal xₐ(t) is contaminated by noise v(t) from the collisions of the airflow onto the airway, electronic devices, and ambient noise. This noise must be removed from the captured lung sound signal y(t) through denoising/signal processing. However, artifacts may be introduced during lung sound signal denoising. The artifacts can lead to misinterpretation or affect the assessment. For example, the detection of wheeze with different pitches is associated with different clinical conditions as explained in N. Meslier, G. Charbonneau, and J. L. Racineux, “Wheezes,” European Respiratory Journal, vol. 8, no. 11, p. 1942, 1995, which incorporated herein by reference. As for crackle, the wrong estimation of the initial deflection width (IDW) and two-cycle duration (2CD) can lead to an inaccurate or incomplete diagnosis as explained by D. F. Ponte, R. Moraes, D. C. Hizume, and A. M. Alencar, “Characterization of crackles from patients with fibrosis, heart failure and pneumonia,” Medical Engineering & Physics, vol. 35, no. 4, pp. 448-456, 2013 Apr. 1, which is incorporated herein by reference.

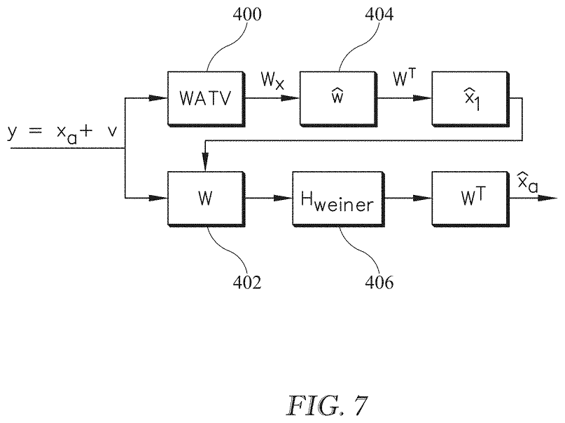

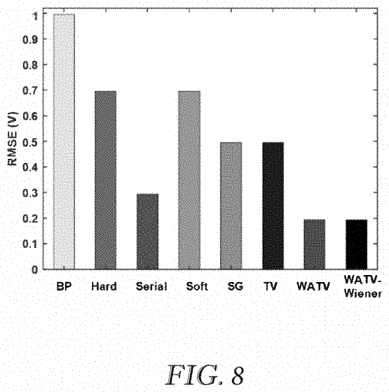

Referring to FIG. 7, to establish an empirically based Wiener filter, WATV 400 was applied to a noisy signal y(n) with wavelet transform W 402 and a single objective function \hat{ω} 404 to obtain xₜ(n). The denoised signal xₜ(n) from WATV is employed to design the empirical Wiener filter H 406 to smooth the denoised signal to reduce artifacts and obtain the desired signal xₐ(n). n is denoted as the sample index, and the total number of samples N over a known time T is defined as N=fₛT , where fₛ is the sampling frequency, and set to fₛ=4000 Hz in developing the disclosed embodiment. However, other frequencies may be used as would be understood by those of ordinary skill in the art.

In WATV, a 5-scale undecimated discrete wavelet transform W with two vanishing moments fulfilling the Parseval frame condition with Daubechies filter (due to its translation-invariant property in denoising) is used for denoising signal with a low- and high-pass analysis filter,

Wy(n)=Wxₐ(n)+Wv(n), n=1, 2, . . . N (10)

W is denoted as wavelet transform for denoising signal in (10). The ‘nonstationary’ region of the lung sound signal produces significant wavelet transform coefficients (amplitude) over many wavelet scales. Most of the significant coefficients at each wavelet scale correspond to the desired lung sound signals, whereas the insignificant wavelet coefficients with small values, typically noise, are shrunk during denoising. w is denoted as the wavelet coefficients containing the signal xₜ required for the designing of the empirical Wiener filter,

ω=Wxₜ (11)

Thus, the estimation of signal xₜ denoted as \hat{x}ₜ is obtained by inverse wavelet transform of wavelet coefficients ω once the estimated wavelet coefficients \hat{ω} is available according to:

\hat{x}_{t =W}^{T}\hat{ω} (12)

The wavelet coefficients \hat{ω} can be identified as described below. Indexing the terms j and k to represent the scale and time information of the signal in the wavelet coefficients ω_{j,k} respectively. The l₁ norm ∥DW^{T}ω∥₁ is defined as the total variation of signal estimation, where D is the first-order difference matrix. Σₙ|xₙ|, Σₙ|x_{n|}² is represented as the indexed and doubly indexed coefficients ∥x∥₁, ∥x∥₂² respectively. To optimize the recovered signal, the choice of regularization parameters the threshold shape controller αⱼ penalty function ϕ and TV parts β are critical. Appropriate parameters may be chosen according to Y. Ding et al. discussed above.

A split augmented Lagrangian shrinkage algorithm (SALSA) is applied to solve the WATV denoising problem:

$$\hat{ω} ( n ) = arg {min}_{ω} \{ F ( ω ) = \frac{1}{2} {‖ Wy - ω ‖}_{2}^{2} + {\sum }_{j , k} {λ}_{j} ϕ ( {ω}_{j , k} ; {α}_{j} ) + β {‖ {DW}^{T} ω ‖}_{1} \} . \qquad ( 13 )$$

To achieve a balance between wavelet-based and TV denoising, they are controlled by a parameter η, and a value of 0.9 has been found to be suitable in one empirical analysis. Thus, the regularization parameter is given as λⱼ=2.5ησ/2^{j/2} and TV parts β=(1−η)(√N)/4σ, where σ=3, is the standard deviation of noise in each wavelet scale j. From the regularization parameter above, the threshold shape controller is identified as α=1/λⱼ.

The estimated denoised signal \hat{x}ₜ is applied into empirical Wiener filter design in (14)-(15) for smoothing and eliminates the artifacts by minimizing the root-mean-squared error (RMSE) to design an improved weighting profile in (15), where σ² is the noise variance from the wavelet transform as expressed in (14) and (15).

$${\hat{x}}_{a} = {W}^{T} H W {\hat{x}}_{t} , \qquad ( 14 )$$

$$H = \frac{{\hat{x}}_{t}^{2}}{{\hat{x}}_{t}^{2} + {σ}^{2}} . \qquad ( 15 )$$

The full technique is summarized in FIG. 7. A good denoised signal is achieved (low RMSE); however, minimal defects may persist. Thus, a hybrid WATV and wavelet-based empirical Wiener filtering to smoothen the denoised signal further to achieve a better-denoised signal in terms of both SNR and RMSE is used.

From FIG. 7 and (10)-(15), applying the estimated denoised signal x₁(n) from WATV to obtain an adequate signal estimate instead of deciding on two wavelet transform bases to obtain an optimal empirical Wiener filter. The pseudocode of the implemented algorithm is shown below:

Input: Noisy data (y); Number of vanishing moment (k); Regularization parameter (λⱼ);
TV parts (β); Step size (μ); Number of wavelet scale (j); Number of iteration (niter).
Initialization: u = Wy; d = u; αⱼ = 1/λⱼ;
//Iteration till convergence
For i = 1; niter
p = [Wy + μ(u − d)]/(1 + μ)
//Finding the output threshold of ω for all j, k with the information from p, λⱼ, μ, αⱼ = 1/λⱼ
ω_{j,k} = p_{j,k}; λⱼ/(1 + μ); αⱼ
v = d + ω
//Total variation denoising (tvd) requires data input, length of the data input (N) and TV
parts
d = W[W^{T}v − tvd(W^{T}v, N, β/μ)]
u = v − d
End For
Output: xₜ = W^{T}ω
//Empirical Wiener filter design for smoothing: H
\hat{x}_{α} = W^{T}HW\hat{x}ₜ
//Supply x₁ into the filter design
H = \hat{x}ₜ²/(\hat{x}ₜ² + σ²)

The acoustic signal captured by one or more sensors 210 is processed to output airflow intensity like that obtained from a pulmonary function test, a lung clearance index, or a patient-based report. In further embodiments, the acoustic signal captured by one or more sensors 210 may be processed to output a regional airflow intensity. In some embodiments, acoustic signal captured by one or more sensors 210 may be processed to provide caregivers with regional/localized information in the form of an image output. In additional embodiments, acoustic imaging captured by one or more sensors 210 may enable caregivers to assess early symptoms and reduce therapy time by providing information focused on the affected chest area. Thus, assessment of lung function done by capturing an acoustic signal by one or more sensors 210 may provide an output that overcomes clinical challenges faced by other imaging techniques such as CT imaging, chest X-rays, MRI, and EIT. Nevertheless, in some embodiments, the output obtained by processing the acoustic signal captured by one or more sensors 210 may be one or images that resemble the analysis of images obtained by other imaging methods such as EIT, radiography, or ultrasound.

The lung function assessment and therapy's effectiveness may be discerned by evaluating acoustic signal source location's pathological abnormalities at two different time points. In some embodiments, the lung function assessment and therapy's effectiveness may be discerned by evaluating the output in the form of one or more images obtained at two different time points. In some further embodiments, the lung function assessment and therapy's effectiveness may be discerned by evaluating the output in the form of regional intensity values obtained at two different time points.

In one embodiment, the sensor array 122 of the sensing unit 120 may have 40 sensors and produce results comparable to VRI. In other embodiments, the sensor array 122 may have a different number of sensors 210 and still produce results comparable to VRI. The sensor array 122 may be arranged to produce images from either the right lung, the left lung, both lungs, or a particular portion of interest of a patient's airways.

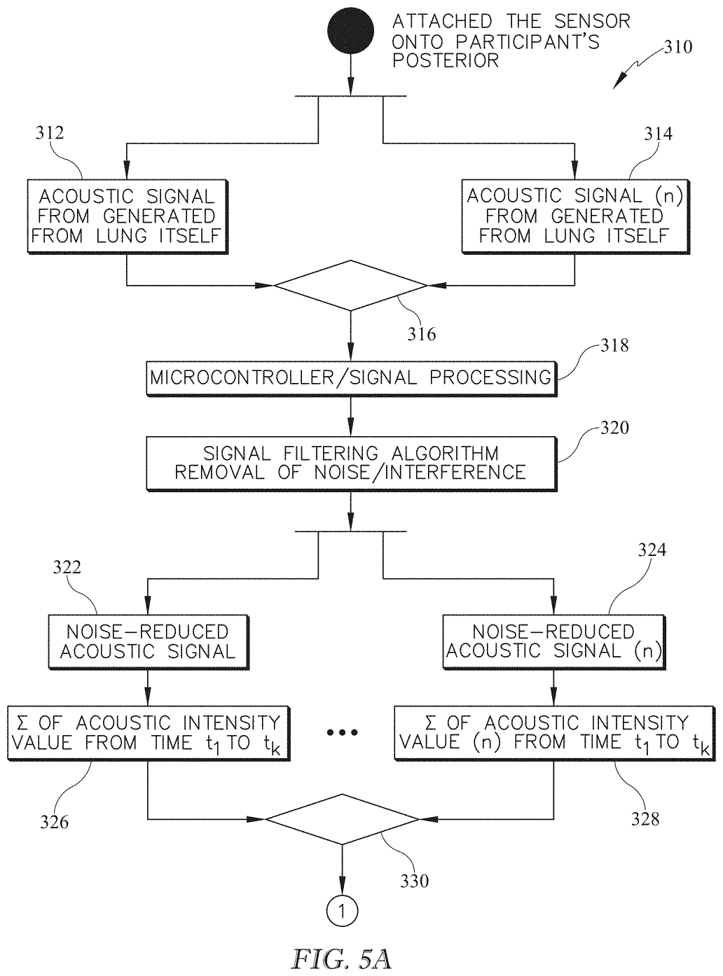

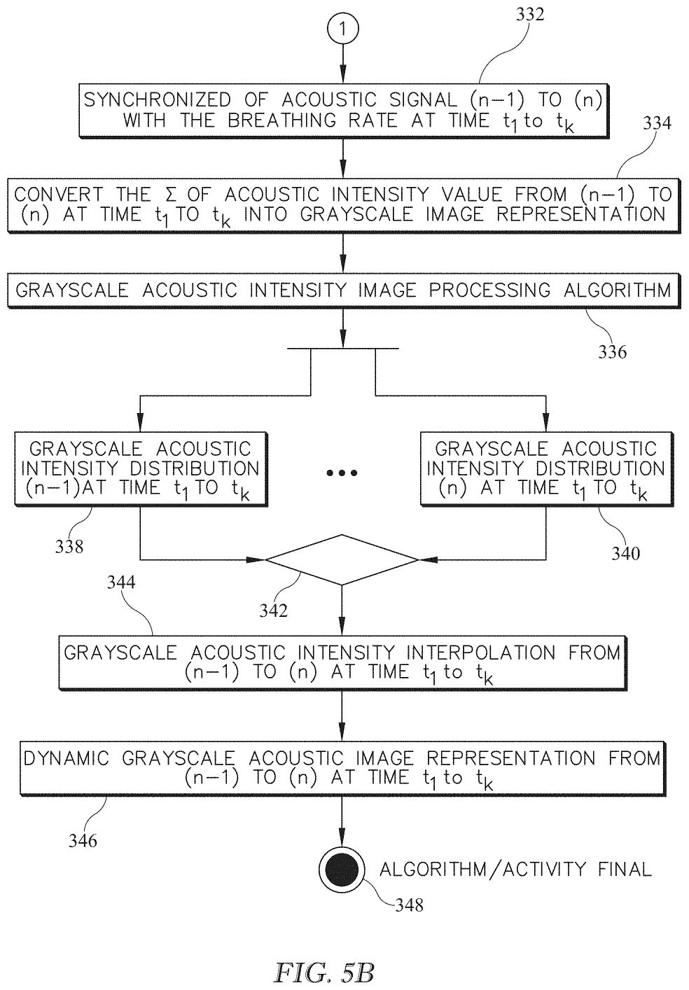

In an alternative embodiment, FIGS. 5A-5B illustrate a process 310 of generating dynamic graphical representations of the acoustic signals captured by a single sensor 210 which is moved to different locations on a patient 110 over a given time interval. The single sensor 210 captures a first acoustic signal at step 312. Once the signal is captured at step 312, multiple additional signals (n) are captured as suggested by step 314. In this case, (n) is a positive integer greater than 1 and represents the total number of positions that a single sensor 210 is placed to capture the acoustic signal from the lung(s) of a patient over a given time interval from t₁ to t₂. In the algorithm 310, the process steps presented on the left are those steps for the first position of the sensor 210 and those on the right represent the repeating of the steps on the left for (n) cycles to establish an image from a single sensor 210.

The (n) signals captured at step 312, 314 are then combined and processed by a microcontroller in step 318 before a signal filtering algorithm is applied to the processed signal to remove any noise or interference in step 320. A denoising filter is applied to the captured signals at step 322, 324 to produce a noise-reduced acoustic signal for the particular position (n). The denoising filter allows only the desired frequency range of respiratory sounds. Thus, the average acoustic intensity is computed over a given time interval from t₁ to t₂ at step 326, 328.

In one embodiment, filtering at step 322 or 324 may be accomplished by a hybrid approach of signal processing that utilizes wavelet-based total variation and a Wiener filter to filter adventitious sounds from a lung sound signal to provide a more accurate assessment as described below

The processed acoustic signals are then synchronized with the patient's breathing rate in step 332. The acoustic signal taken from (n−1) to (n) is synchronized with the breathing rate at time t₁ to tₖ. The acoustic intensity value determined from (n−1) to (n) at time t₁ to tₖ is converted into a grayscale image in step 334. The grayscale intensity image is them processed using an image processing algorithm in step 336 to generate a grayscale acoustic intensity distribution (n−1) at time t₁ to tₖ in step 338 and a grayscale acoustic intensity distribution (n) at t₁ to tₖ in step 340. The images generated in steps 338 and 340 are merged in step 342.

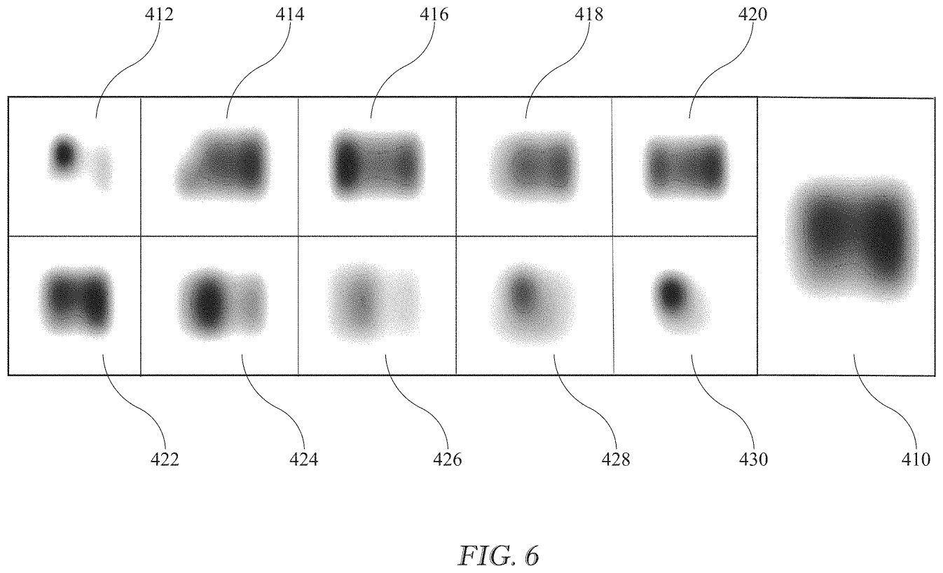

The acoustic intensity at any location nᵢ is estimated using an interpolation method in step 344 to generate a dynamic grayscale acoustic image 410 in step 346 representing the acoustic signals from (n−1) to (n) at time t₁ to tₖ as shown in FIG. 6, where the grayscale images 412, 414, 416, 418, 420, 422, 424, 426, 428 and 430 represent the acoustic intensity 420 at each point in time t₁ to tₖ.

In one illustrative embodiment, as shown in FIG. 4, the signal processing unit 130 used to process and convert an acoustic signal captured from a patient's lung to an output representing function of the lung is performed by a computing device 510. The computing device 510 may be embodied as any type of computation or computer device capable of performing the functions described herein, including, but not limited to, a server (e.g., stand-alone, rack-mounted, blade, etc.), a network appliance (e.g., physical or virtual), a high-performance computing device, a web appliance, a distributed computing system, a computer, a processor-based system, a multiprocessor system, a smartphone, a tablet computer, a laptop computer, a notebook computer, and a mobile computing device.

In one embodiment, the computing device 510 includes an input/output (I/O) subsystem 502, a memory 504, a processor 506, a data storage 508 device, a communication subsystem 512, a signal processing unit 130, and a display 514. The computing device 510 may include additional and/or alternative components, such as those commonly found in a computer (e.g., various input/output devices), in other embodiments. In other embodiments, one or more of the illustrative components may be incorporated in, or otherwise form a portion of, another component. For example, the memory 504, or portions thereof, may be incorporated in the processor 506.

The processor 506 may be embodied as any type of processor capable of performing the functions described herein. For example, the processor 506 may be embodied as a single or multi-core processor(s), digital signal processor, microcontroller, or other processor or processing/controlling circuit. The memory 504 may be embodied as any type of volatile or non-volatile memory or data storage capable of performing the functions described herein.

In operation, the memory 504 stores various data and software used during operation of the computing device such as operating systems, applications, programs, libraries, and drivers. The memory 504 is communicatively coupled to the processor via the I/O subsystem 502, which may be embodied as circuitry and/or components to facilitate input/output operations with the processor, the memory, and other components of the computing device.

For example, the I/O subsystem 502 may be embodied as, or otherwise include, memory controller hubs, input/output control hubs, sensor hubs, host controllers, firmware devices, communication links (i.e., point-to-point links, bus links, wires, cables, light guides, printed circuit board traces, etc.) and/or other components and subsystems to facilitate the input/output operations.

In one embodiment, the memory 504 may be directly coupled to the processor 506, for example via an integrated memory controller hub. Additionally, in some embodiments, the I/O subsystem 502 may form a portion of a system-on-a-chip (SoC) and be incorporated, along with the processor 506, the memory 504, and/or other components of the computing device 510, on a single integrated circuit chip.

The data storage device 508 may be embodied as any type of device or devices configured for short-term or long-term storage of data such as, for example, memory devices and circuits, memory cards, hard disk drives, solid-state drives, or other data storage devices. The computing device 510 also includes the communication subsystem 512, which may be embodied as any communication circuit, device, or collection thereof, capable of enabling communications between the computing device 510 and other remote devices. The communication subsystem 512 may be configured to use any one or more communication technology (e.g., wired or wireless communications) and associated protocols (e.g., Ethernet, InfiniBand®, Bluetooth®, Wi-Fi®, WiMAX, 3G, 4G LTE, 5G, etc.) to effect such communication.

The display of the computing device 514 may be embodied as any type of display capable of displaying digital information, such as a liquid crystal display (LCD), a light emitting diode (LED), a plasma display, a cathode ray tube (CRT), or other type of display device. In some embodiments, the display 514 may be coupled to or otherwise include a touch screen or other input device.

As illustrated in FIG. 4, the image processing unit 140 is in the same computing device 510 as the signal processing unit 130. In other embodiments, the image processing unit 140 may be in a different computing device. The computing device 510 may also include any number of additional input/output devices, interface devices, hardware accelerators, and/or other peripheral devices.

In an effort to optimize the overall filter performance, simulations modifying three parameters η, σ, and N that influence the TV parts β and regularization parameter λⱼ. Referring to equation (13), both β and λ control the pilot estimation of the denoised wavelet coefficients. As the pilot estimation affects the designing of the empirical Wiener filter and the overall filter performance, the pilot estimation of the denoised wavelet coefficients is important to establishing the filter. To evaluate the effect of the parameter η on the overall filter, the denoised noisy lung sound signals in the SNR sense were compared to determine the performance.

Three possible simulation case studies were evaluated through adjusting η, while keeping σ=10 and the total number of samples N=4000. In a first simulation, η was maintained at 0.76<η<1, e.g., η=0.80, η=0.90, which resulted in Σλⱼ>β. In a second simulation, η was maintained at 0<η<0.76 to a lower value, e.g., η=0.2, 77=0.5, resulting in β>Σλⱼ. Final, as simulation was run at β≈λⱼ with η=0.76. The mean SNR improvement with respect to the ratio between TV parts and regularization parameter in the three explored scenarios were established. The SNR performance of the filter is at the lowest when β>Σλⱼ, with a ratio <1, and achieved best SNR performance when Σλⱼ=3β, at η=0.90.

Through this evaluation, the condition η=0.90, was established as a baseline for optimizing the SNR performance. Additionally, the TV parts β=(1−η)(\sqrt{N})/4σ and the regularization parameter λⱼ=2.5ησ/2^{j/2} were also determined by the total number of sample N. Typical lung sound signals are comprised of minimally two respiratory cycles with time T≈4s, Fₛ=4000 Hz. Additional analysis was conducted by adjusting the total number of samples N to actual lung sound signals to determine if N affects the overall filter performances. The performance of the WATV-Wiener filter was not affected by the total number of samples in terms of SNR performance and showed a similar SNR performance trend at the ratio Σλⱼ=3β, with η=0.90 regardless of number of signal samples.

As η ranges between 0 and 1, 0.9<η<1 has been identified as a baseline from the discussion above. To optimize of parameter η from the baseline parameters identified from empirical data, different possible combinations of parameter η, e.g., η=0.90, η=0.95, and η=0.99 were set to evaluate the optimal SNR performance the overall filter on different noisy lung sound signals. WATV-Wiener filter obtained higher improved SNR by 3-8 dB with η=0.95 compared to the prior art appraches estimated parameter, and the initial investigation η=0.90, and the SNR performance is similar for both η=0.95 and η=0.99, with a variation of 1 dB. In addition, WATV-Wiener performed better in terms of SNR with the single setting of Σλⱼ>β with 0.95≤η<1 compared to the other case settings.

It has been determined that optimal SNR results following optimized parameters for denoising typical lung sound signals by tuning Σλⱼ=3β with 0.95≤η<1. Ultimately, the denoised signal ought to retain waveform characteristics of interest without overly deforming the lung sound signals. Thus the ideal parameter 0.95≤η<1 is used denoise noisy lung sound signals with different noise variances and achieved consistent RMSE results with different noise variance, showing robustness to the noise variance.

Inappropriate selection of parameter, e.g., in the wavelet thresholding, may result in a high SNR, albeit evident artifacts are introduced in the signal processing. Thus, RMSE is likewise an important factor to identify that the denoising filter retains the frequencies of interest and waveform characteristics. In the prior art, WATV is an optimal denoising filter in the RMSE sense. However, it is important to denoise the signal without affecting the waveform characteristics while improving the SNR. Using the parameters identified in the optimal tuning study, 0.95≤η<1, the WATV-Wiener filter was compared with other established lung sound signal filters in the prior art. The simulated lung sound signals have the following parameters: noise variance σ²=9, Fₛ=4000 Hz, and the total number of samples N=16000.

WATV denoising filter is optimal in terms of RMSE, achieving mean RMSE of adventitious lung sound signals of 0.43 V in crackle, 0.47 V in wheeze, 0.21 V in healthy lung sounds. WATV-Wiener shadows WATV sharply, within ±0.02 V, or within 10% in the absolute relative change in terms of optimal RMSE, and performed better by 0.2-0.7 V compared to remaining filters, i.e., BP, Soft, Hard, Serial, and TV. WATV-Wiener obtained the best mean improved SNR of 38.09±0.80 dB in crackle, 41.03±0.79 dB in wheeze, and 47.56±0.73 dB in healthy lung sound signals. From the results in SNR and RMSE, the BP filter has the lowest SNR performance and worse RMSE results. This is possibly due to denoised lung sound signals containing overlapping noise spectral. The finding in RMSE is consistent with the prior art where a single linear infinite impulse response or FIR-based filter may not be sufficient to denoise a noisy signal, and the noise affects the waveform characteristics.

From the SNR and RMSE results, WATV-Winer filter can achieve optimal RMSE results similar to the optimal RMSE-sense WATV, and further achieved higher noise removal in terms of SNR by another 5-20 dB compared to other established lung sound signals filters. From the RMSE and SNR results, WATV-Wiener showed it could retain waveform characteristics (low RMSE) while improving SNR from denoising various inputs of SNR lung sound signals, showing robustness to severe noise. The WATV-Wiener performance benefits achieved could be due to the optimized pilot estimation of the wavelet coefficient \hat{ω} and smooth the pilot denoised wavelet coefficient with the complementing diagonal weighting matrix H from empirical wiener filter. Without the optimal parameters in the pilot estimation of the denoised wavelet coefficient, the integration of the WATV filter and empirical Wiener filter may not achieve optimal denoised lung sound SNR performance. WATV estimates the wavelet coefficients \hat{ω} by considering both insignificant (noise) and significant (signal) coefficients. The disclosed approach uses the estimated signal estimates from WATV to design an empirical Wiener filter H to smooth and reduce the artifacts on the denoised signal. The empirical Wiener filter scales the coefficients by minimizing the MSE to design an improved weighting profile H≈1, with a WATV coefficient more significant than the noise variance, \hat{ω}>>σ². Thus, pilot estimation of the denoised wavelet improves the weighting profile of the filter, and this disclosed proposed hybrid technique can decrease the denoised signal's bias and achieve an optimal filter in SNR performance. Under the condition of the noise variance σ² is greater than the estimated denoised signal \hat{ω}², the weighting profile will contribute to the gain in wavelet coefficient resulting in a lower SNR performance.

To ensure the denoising performance stability of the WATV-Wiener filter between simulation studies and actual respiratory sound, a quantitative comparison of the WATV-Wiener filter and other prominent filters in the prior art was performed. Ten healthy lung sound signals from volunteers with was used to evaluate the system SNR performance compared against a commercial product used for capturing lung sound signals. Experiments were performed in an uncontrolled environment with an average 59±0.54 dBA sound pressure level, similar to a hospital noisy intensive care unit, where emergency alarm, communications, and critical care are often happening.

Unhealthy lung sound signals containing crackle, wheeze, or both crackle and wheeze (mixed) were selected from an open-access respiratory database. The respiratory database captured volunteers' respiratory sounds by digital stethoscope or an array of MEMS microphones in a clinical or home setting, with qualified independent reviewers annotating the signals. The signals also contain cough, speech, and throat clearing. The unhealthy respiratory signals have a minimum sampling frequency of Fₛ=4000 Hz, and a minimum recording time of T=10 s. A total of 50 recordings from captured healthy lung sound signals and the unhealthy respiratory signals were passed through the denoising filters to estimate the denoised signal's SNR output.

Before denoising, a bandpass filter ranging 150 Hz-1300 Hz was applied to remove other major artifacts events such as cough and throat clearing. All patients in the respiratory signal database had chronic obstructive pulmonary disease (COPD) with comorbidities—heart failure. Hence, signals below 150 Hz are excluded. A maximum of 1300 Hz was chosen as the upper bandpass limit as Fₛ=4000 Hz to avoid aliasing effects. In the prior art, healthy, wheeze, and crackle frequency signal falls within the bandpass range of 150 Hz and 1300 Hz; confirming that it is sufficient to retain the interest frequency range and adventitious lung sound characteristics after denoising.

The estimated noise variance is about σ²=0.05(σ=0.23) from healthy lung sound signal measurement in experimental studies. The lung sound signals were resampled with a sampling frequency of Fₛ=4000 Hz, and applied σ=0.23, and the optimal parameter evaluated from the simulation studies, η=0.95 to the experiment analysis as the sound pressure level for capturing the healthy lung sound signals and the database is similar. The static and ambient noise in the database may be different from captured lung sound signals. The WATV-Wiener filter is insensitive to noise variance in simulation studies, achieving similar SNR and RMSE performance in both low and high noise variance with 0.95≤η<1.

RMSE_{system}=\sqrt{mean[(d−x)²]}, (16)

where d denotes the amplitude of denoised lung sound signals, x represents the amplitude of noise-free lung sound signals given in (22),

x=aₛ−aₙ, (17)

where the captured lung sound signals with noise and captured ambient noise without lung sound signals are denoted as aₛ and aₙ, respectively.

The computation of SNR in (23) and (24) is similar to (14), defining SNR by finding the ratio of denoised signal peak amplitude to noise peak amplitude and expressing the ratio using the logarithmic decibel scale.

$$S N {R}_{s y s t e m} = 20 [ log 10 ( \frac{d}{{a}_{n}} ) ] , \qquad ( 18 )$$

where aₙ is the noise peak amplitude from the system electronic static noise and ambient noise without lung sound signals, and d denotes the filter denoised signal peak amplitude.

There is a slight modification for the computation of database SNR in (24) as noise is unavailable; thus, ‘noise’ is defined as subtracting the denoised signal from the noisy signal:

$$S N {R}_{database} = 20 [ log 10 ( \frac{d}{{f}_{y} - d} ) ] , \qquad ( 19 )$$

where, f_{y} is the noisy signal peak amplitude from the database, and d is the denoised signal peak amplitude.

An acoustic sensor-based MEMS for capturing lung sounds was used. The system included a high SNR microelectromechanical system (MEMS) microphone with a frequency response between 50 Hz and 20 kHz. The sampling frequency of the MEMS microphone is 44100 Hz, and the MEMS sensor consists of a signal conditioning function, an analog-to-digital converter, decimation and anti-aliasing filters, power management, and an industry-standard 24-bit time-division multiplexing interface. A 3M electronic stethoscope has ‘proprietary’ ambient noise reduction technology that eliminates an estimated 85% of ambient background noise interference without eliminating critical lung sounds. A study was conducted comparing the disclosed system performance against the 3M electronic stethoscope. The SNR computation for the MEMS system and 3M electronic stethoscope can be expressed as SNR=20 log(āₛ/āₙ), where, āₛ=Σ(aₛ−aₙ)/N represents the mean peak amplitude of the signal, and āₙ=Σaₙ/N is the mean peak value of the collected noise without lung sound signal from the MEMS sensor and 3 M electronic stethoscope. aₛ is the peak amplitude of the collected lung sound signal with noise, aₙ is the peak value of the collected noise without lung sound signal, and N=10 is the number of collected signals. An estimated SNR of 71.63 dB for the MEMS system can be compared to 68.73 dB for the 3 M electronic stethoscope. Thus, the disclosed system performs similarly to the commercial 3 M electronic stethoscope in terms of SNR.

The disclosed WATV-Wiener achieved a similar optimal RMSE of 0.1933 V compared to the optimal WATV filter in the RMSE sense at 0.1938 V, achieving an absolute relative change of about 0.26%. A similar trend from the simulation studies, particularly in the BP filter, where noise presence in the overlapped spectral may affect the overall filter signal quality, resulting in a high RMSE result of 0.99 V. Altogether, the WATV-Wiener filter achieved better and optimal RMSE results by about 0.1-0.8 V as compared to other filters such as BP filter, Hard filter, Serial filter, Soft filter, SG filter, and the TV filter.

Further evaluation of denoising filter performance shows that the WATV-Wiener filter improved SNR by about 4-30 dB compared to other denoising filters, consistent with the simulation study findings (5-20 dB). As noise might be present in the denoised signal from BP filter, WATV-Wiener improved SNR by about 44 dB in healthy lung sound signals, consistent with the SNR results in the simulation studies.

It is known that denoising continuous piecewise signal, e.g., healthy, and wheeze is more straightforward than denoising noncontinuous piecewise signal, e.g., crackle; however, similar performance in terms of improved SNR, about 49 dB between crackle and wheeze has been achieved. From the experimental results, the WATV-Wiener filter functions better than the WATV filter in denoising noisy signals achieving optimal RMSE, and improving the SNR, and the noise variance has minimal effect on WATV-Wiener.

Better (optimal) RMSE results by 0.1-0.8 V in the actual healthy lung sound signals than other filters was achived, similar to simulation studies (0.2-0.9 V). Similar improved SNR for healthy lung sound signals was achieved between the simulation studies and experiment at about 3-20 dB with the optimal parameters. The improved SNR of about 4-30 dB was attained for adventitious (crackle, wheeze, mixed) lung sound signals. The improved SNR in experimental studies (4-30 dB) is higher than the improved SNR in the simulation studies (5-20 dB) could be attributed to the modified computation of SNR as shown in (24), where noise is defined as the differences between denoised lung sound signals and observed noisy lung sound signals. Although a difference of 10 dB in improved SNR for crackle and wheeze is observed between experiment and simulation studies, the minimal difference can be further reduced if noise data is available in the database. ‘Noise data’ is typically available in practice and usually referred to using a sensor to capture the static electronic interference and ambient noise without lung sound signals similar to the system's captured noise aₙ in (23), and in the prior art.

The optimal results obtained by the WATV-Wiener filter could be: 1) due to the advantage of wavelet-based denoising in noncontinuous piecewise signal, and 2) the optimal integration of two ideal filters, particularly in the RMSE sense, by addressing different challenges faced individually, e.g., WATV eliminates the requirement of selecting two different wavelet transform bases compared to empirical Wiener filter, but introduces artifacts, and empirical Wiener filter (known for eliminating artifacts through minimizing MSE) to remove the artifacts introduced by WATV.

A further use of the lung imaging described herein is with spatially distributed airway tree models to decipher the relationship between bronchi lengths, branching angles, and airway diameters. In the development of the models, Murray's law defines that the relationship between airway bifurcation is fixed, with branch lengths based on a length-to-diameter ratio. Weibel symmetric and Horsfield asymmetric models are the most used conducting airway models.

With the advancement of medical imaging techniques, deterministic parameterized bronchial tree generation algorithms were extracted directly from CT, thus constituting the core of patient-specific modeling. However, the models developed so far are typically simplified to a one-dimensional system of equations to investigate the relationships between healthy and unhealthy respiratory system cycles, such as frequency response, flow rate, resistance, volume and the diagnosis accuracy. The development of respiratory airway modeling and the acoustic imaging is disclosed below.

Based on the developments to date, a model for acoustic imaging to enhance the study on optimizing HFCWO targeted therapy has been developed. The models is a two-dimensional (2D) model required for lung imaging and demonstrates obstruction and the localization of nidus in the airways. Using the techniques described above with the model described below, the resolution of the lung image was intended mainly for the assessment and localization of nidus in the airways due to the limited number of sensors and HFCWO actuators.

In the model development, the following notations were used. R denotes the set of all real numbers. R^{m×n} is the set of all real (m×n) matrices. C denotes the set of all complex numbers. C^{m×n} is the set of all complex (m×n) matrices. Z(ω) is the set of all sinusoidal variables with angular frequency co. The airway wall Young's modulus and material density were employed to replicate the acoustic structural interaction accurately.

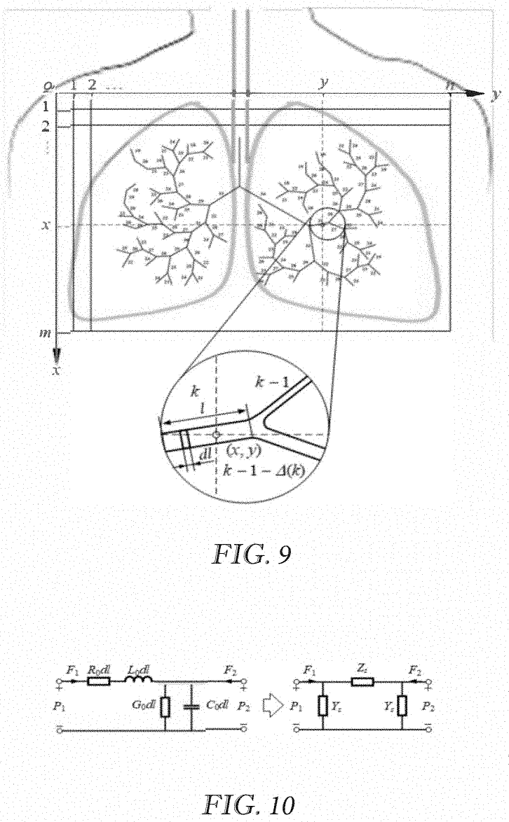

Each 3-dimensional (3D) network segment is first projected to a 2D plane and given a location coordinate (x, y). Then, the respiratory system is represented as a bifurcating tree network, where the joined node of the bifurcating segment at layer k and location (x, y) is indexed by (x, y, k) on the plane shown in FIG. 9. The k-th layer segment bifurcating into asymmetry airways of layers (k+1) and (k+1 +Δ(k)) through a recursion index Δ(k). Next, the airway is modeled as a network of bifurcating cylinders and can be converted into an electrical π network with lumped parameters presented in FIG. 9 and FIG. 10, respectively. After that, the acoustic pressure at each segment caused by the pressure distribution from bronchi breathing along with the airway network is resolved by the grapy network hypothesis. Lastly, the projected network is a subset of the acoustic lung image F(x,y)∈R^{m×n} by combining the acoustic power over a set amount of time throughout each breathing cycle.

When the patient breaths smoothly, it can be considered a steady system with the fundamental angular frequency ω of the respiratory frequency. Each segment can then be considered as a transmission line with parameters R₀, L₀, C₀, G₀ corresponding to a small nonrigid tube with analogous acoustic resistance, inductance, capacitance, the conductance of the tube, respectively, which is described by (20),

$$( \begin{matrix}{P}_{1} \\ {F}_{1}\end{matrix} ) = ( \begin{matrix}cosh ( γ l ) & {Z}_{c} sinh ( γ l ) \\ \frac{1}{{Z}_{c}} sinh ( γ l ) & cosh ( γ l )\end{matrix} ) ( \begin{matrix}{P}_{2} \\ {F}_{2}\end{matrix} ) , \qquad ( 20 )$$

where P₁∈Z, F₁∈Z, P₂∈Z, F₂∈Z are input pressure, input flowrate, output pressure, and output flowrate, respectively. γ∈C is the propagation coefficient and Z_{c}∈C is the characteristic impedance given in (21).

$$\{ \begin{matrix}γ = \sqrt{( {R}_{0} + j ω {L}_{0} ) ( {G}_{0} + j ω {C}_{0} )} \\ {Z}_{c} = \sqrt{( {R}_{0} + j ω {L}_{0} ) / ( {G}_{0} + j ω {C}_{0} )}\end{matrix} . \qquad ( 21 )$$

The lumped parameters of segment impedance Zₛ∈ [symbol] and segment admittance Yₛ∈ [symbol] are given in (3).

$$\{ \begin{matrix}{Z}_{s} = {Z}_{c} sinh γ l \approx ( {R}_{0} + j ω {L}_{0} ) l \\ {Y}_{s} = \frac{cosh γ l - 1}{{Z}_{c} sinh γ l} \approx \frac{1}{2} ( {G}_{0} + j ω {C}_{0} ) l\end{matrix} . \qquad ( 22 )$$

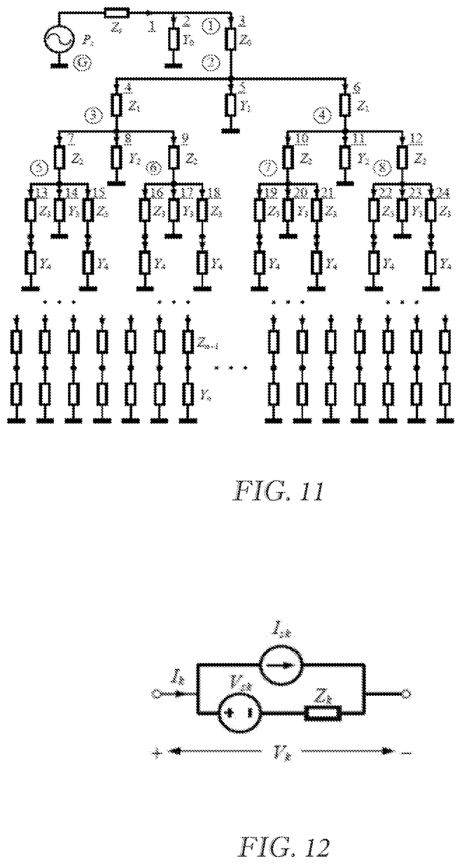

Therefore, the whole airway network can be modeled as an electrical network constructed by a layered bifurcating tree of impedance with an admittance at each bifurcating node standing up to the ground, as shown in FIG. 11. In analyzing the respiratory airways as an electrical network, the air pressure and airflow rate are equivalent to electrical potential and current, respectively. The impedance and admittance in the k-th layer can be presented in (23).

$$\{ \begin{matrix}{Z}_{k} = {Z}_{s} ( k , ω ) \\ {Y}_{k} = {Y}_{s} ( k , ω ) + 2 {Y}_{s} ( k + 1 , ω )\end{matrix} , k = 0 , n . \qquad ( 23 )$$

If the network has n nodes and b branches, a sinusoidal voltage source with amplitude Pₛ and angular frequency ω is applied at the input layer 0 to represent the fundamental component of the periodical patient breath. From the theory of network topology, the following annotation is applied:

A∈R^{(n-1)×b}, Y∈C^{b×b}, V∈Z^{b×1}, I∈Z^{b×1},

Y_{b}∈C^{b×1}, Vₙ∈Z^{n×1}, Vₛ∈Z^{b×1}, Iₛ∈Z^{b×1},

where A, Y, V , I , Y_{b}, Vₙ, Vₛ, and Iₛ are reduced incidence matrix, branch admittance matrix, branch voltage vector, branch current vector, branch admittance vector, and node potential vector, branch voltage source vector, and branch current source vector, respectively. FIG. 10 shows a standard branch in a linear network, and the node analysis approach can be given as (24),

$$\{ \begin{matrix}A \cdot I = 0 \\ {A}^{T} \cdot {V}_{n} = V \\ I = Y \cdot V + {I}_{s} - Y \cdot {V}_{s}\end{matrix} , \qquad ( 24 )$$

where the first and the second conditions in (24) are Kirchhoff's current law and Kirchhoff's voltage law, respectively. The third condition is derived from the standard branch law, and (24) can be used to obtain (25):

A·Y·A^{T}Vₙ=A·Y·Vₛ−A·Iₛ, (25)

where vₙ is the remaining unknown variable. Assuming Yₙ∈C^{(n-1)×(n×1)} is a nonsingular and symmetric node admittance square matrix and Jₛ∈Z^{(n-1)×1} is the node source-current vector as shown in (26). Vₙ can be resolved in (27):

$$\{ \begin{matrix}{Y}_{n} = A \cdot Y \cdot {A}^{T} \\ {J}_{s} = A \cdot Y \cdot {V}_{s} - A \cdot {I}_{s}\end{matrix} , \qquad ( 26 )$$

$${V}_{n} = {Y}_{n}^{- 1} \cdot {J}_{s} . \qquad ( 27 )$$

In FIGS. 11 and 12, the nodes are indexed by encircled numbers and the branches by underlined numbers. We have b=3×2ⁿ, Iₛ=0 and the reduced incidence matrix and branch admittance matrix are denoted as follows:

$$A = ( \begin{matrix}{A}_{11} & {A}_{12} \\ {A}_{21} & {A}_{22}\end{matrix} ) , \\ Y = diag ( {Y}_{b} ) , \\ {V}_{s} = {[ {P}_{s} {0}^{1 \times ( b - 1 )} ]}^{T} {A}_{11} = [ 1 - 1 ] , \\ {A}_{12} = [ 1 {0}^{1 \times ( b - 3 )} ] , {A}_{21} = {0}^{( n - 2 ) \times 2} , \\ {A}_{22} = [ {a}_{i , j} ] {\vert}_{\begin{matrix}i = 2 , … , n \\ j = 3 , … , b\end{matrix}} = \{ \begin{matrix}- 1 , if j = 3 ( i - 1 ) + k , k = 1 , 2 , 3 \\ 1 , if j = i + floor ( i / 2 ) \\ 0 , else\end{matrix} .$$

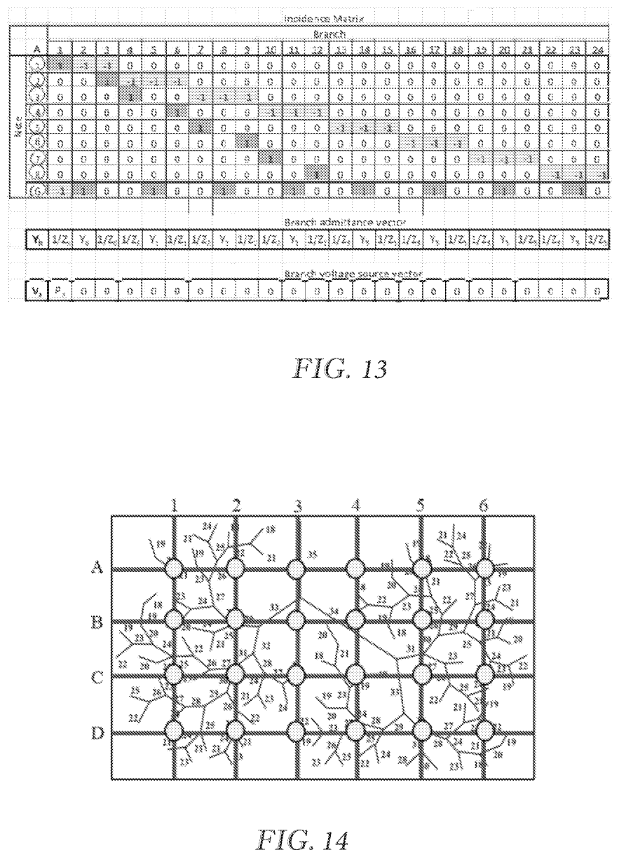

The tables in FIG. 13 shows the indecent matrix, branch admittance vector and branch voltage source vector of the first four layers of the network in FIGS. 11 and 12. The reduced incidence matrix is the matrix without the row of node G. Reference to the Appendix for the respiratory airway modeling with the material properties.

The prior art has investigated the variable physical characteristics, but no spatial information is associated with the nodes, particularly the node voltage. In this disclosure, the spatial location (x, y) is added to each node to generate the acoustic image to transform the airway system into a spatial network. Once the node voltage Vₙ(x, y, t), which is equivalent to the acoustic pressure distribution within the airways Pₙ(x, y, t) , is obtained, the acoustic image is generated in the following way. First, the sound intensity can be computed with (28),

P(x,y,t)=20 log₁₀(Pₙ(x,y,t)/P₀), (28)

where P₀=20 μPa is the reference sound pressure. Let N be the number of sensors and fixed at a position (i=1, 2, . . . , N) to capture acoustic intensity signals P(x, y, t), where (x, y) is the position of the sensor. Next, the average sound intensity over a given time interval t from t₁ to tₖ at each sensor position is computed by averaging the acoustic intensity signal at all nodes within the sensing area as presented in (29):

$$\overline{P} ( x , y , {t}_{1 ′} {t}_{k} ) = \frac{{{\sum }_{{t}_{1}}}^{{t}_{k}} {P}^{2} ( x , y , t )}{{P}_{b}} , \qquad ( 29 )$$

where P_{b} is the number of pressure signal at a specific sensor area as demonstrated in FIG. 14. The projected network of the acoustic lung image F(x, y, t₁, tₖ) is presented in (30),

F(x,y,t₁, tₖ)=P̅(x,y,t₁,tₖ)h(x,y,t₁,tₖ) (30)

The vertical and horizontal lines separate the airway geometry with the multiple sensing areas, and the position of the acoustic sensor is denoted with circles. The sound intensity at any location outside the sensor position is estimated using interpolation. From the observation in (28)-(30), the acoustic lung image F(P̅, h) is defined as the 2D acoustic image which comprise acoustic signal P̅(x, y, t₁, tₖ) in (28)-(30) with subinterval k for interpolating, given as P̅ₖ≤P̅≤P̅ₖ₊₁, and interpolation polynomial h(x, y, t₁, tₖ). The Hermite interpolation function was applied to the acoustic signal for mapping acoustic thoracic imaging with high spatial resolution. Since the pixel information is known, the interpolation polynomial can be obtained by

$$h ( x ) = {\sum }_{i = 0}^{d} ( {h}_{i} - 2 {L}_{i}^{′} ( {x}_{i} ) ( x - {x}_{i} ) {L}_{i}^{2} ( x ) {h}_{i} + ( x - {x}_{i} ) {L}_{i}^{2} ( x ) {h}_{i}^{′} ) , \qquad ( 31 )$$

where the prime symbol (′) refers to the derivative function, d is the degree of the polynomial, x is the interpolated intensity output from P̅(x, y, t₁, tₖ) with the axis information, and L is the Lagrange polynomial and is given in (32)

$${L}_{i} ( x ) ▯ {\prod }_{0 < m < n} \frac{x - {x}_{m}}{{x}_{i} - {x}_{m}} . \qquad ( 32 )$$

Finally, the highest, lowest, and in-between values are determined as black, white, and grey. Thus, each acoustic signal is normalized, and acoustic images are then displayed as the output collected from the intensity sound signal as described above.

The model developed was then simulated to assess performance. For conciseness, one frequency, 400 Hz was chosen to present the findings in this disclosure as there is a wide range of lung sound frequencies reported in the literature because of its applicability in respiratory sounds, especially for healthy, asthma, and COPD individuals. The airway wall was modeled using the complex Young's modulus and material density based either on a cadaver or CT image to replicate the acoustic structural interaction accurately. The airway segments' thickness, cartilage, and soft tissue fractions were determined by referring to historical data and identifying the closest Horsfield order segment.

A representation of a 2D coordinate image system is the output. The minimal data area—high airflow resistance in the airway, is designated as white. High data area, where airflow resistance is the lowest in the airway, are shown in dark colors (black). In addition, low data areas, where airflow resistance is present in the airway are indicated in light colors (light gray), while minimum data area is represented in white.

A COPD patient's lung sound signal was shortlisted from a respiratory database and was used as a reference image in the simulation study. The sensors are assumed to be uniformly distributed within a 50 mm distance with a four-by-six array of sensors that captures the lung sound signals simultaneously, and the acoustic response is the average intensity value within the sensing area. With spatial location information (x- and y-axis) added at each bifurcate in FIG. 9, acoustic lung image can be generated with (28)—(32).

Airway remodeling by altering the airway radius to simulate the obstructed airway was performed with the AWT constant in a cross-sectional plane. With the assumption that the airway is constant, the total diameter of each airway can be estimated as D=L+h, where h is the bronchial wall thickness and L is the luminal diameter. The airway area A₀=π(D/2)² and the luminal area A₁=π(L/2)² can be computed with D and L, respectively. Airway wall area (WA) is calculated as (A₀−A₁), and WA %=WA/A₀×100.

In the prior art, the airway dimensions of patients with asthma and COPD were compared, and the airway lumen and wall dimensions were assessed using computed tomography in relation to the severity (mild, moderate, severe) of the condition. Mean WA % increment between 3% and 40% has been identified in the prior art, with 0%<control<3%, 3%<mild<10%, 10%<moderate<30%, and severe condition>30%.

The mean acoustic image intensity (dB) was used as a performance index in this disclosure, where the intensity provides suggestive assessment outcome on the severity of the airway obstructions. As different prior provided different results, such as the increment of WA %, or values of AWT, increment of AWT (factor) was applied to standardized the findings in this disclosure. E.g., for the mean WA % to increase about 11%, from 67.28% to 78.93%, the AWT must increase by a mean factor of 2.34. Lastly, the lumen area between asthma and COPD was fundamentally the same in terms of respiratory disease severity. Hence, no distinction between asthma and COPD is performed in this disclosure.

Natural image signals have substantial dependencies between their pixels, especially when they are close together in space. These dependencies include significant information about the organization of the objects in the visual scene. A more direct method for contrasting the reference and distorted signal structures is the SSIM index. Additionally, SSIM indexing offers quality evaluation from the standpoint of image formation, especially for image components in pixel intensities. The computation of three terms, namely the brightness term, the contrast term, and the structure term, is the basis of the SSIM quality assessment index as shown in (33):

$$SSIM ( x , y ) = \frac{( 2 {μ}_{x} {μ}_{y} + {C}_{1} ) ( 2 {σ}_{x y} + {C}_{2} )}{( {μ}_{x}^{2} + {μ}_{y}^{2} + {C}_{1} ) ( {σ}_{x}^{2} + {σ}_{y}^{2} + {C}_{2} )} , \qquad ( 33 )$$

where , μₓ, μ_{y}, σₓ, σ_{xy}, and C are the local means, standard deviations, cross-covariance, and constant for reference image x and captured image y.

This disclosure uses spatial information of 44 pixels per 10 mm of the lung geometry. Acoustic images of the healthy lung (control), and various respiratory disease severity achieved from altering AWT.

The correlation between healthy and unhealthy acoustic lung image can be identified; a healthy lung has the lowest impedance—smallest resistance in the airway, as observed from (20)-(28); thus, presenting the darkest lung image (high acoustic intensity value) as compared to mild, moderate and severe conditions. In addition, both the airflow and the mean image intensity decreased with the increase of AWT. Although obvious qualitative difference can be spotted with the AWT increasing by a factor of about more than 1.70, the mean image intensity can show the condition of the lung health.

Acoustic images of the right lung images generated from (20)-(32) with various severity. Healthy lung; AWT increased by a factor of about 1.26, 1.44, 1.71, 2.84, and 4.97, respectively.

FIG. 7 shows the comparison of the obstructed airway between the reference lung image, which was converted into an acoustic image from the lung sound signal taken from a respiratory database, and the model acoustic image. The obstructed airway is located along the posterior right middle scapular line (area C2) shown in FIG. 14. Obstructed airway region can be observed from the model acoustic image presented in FIG. 16. The similarity between the acoustic reference image and the model acoustic image is highly related as an SSIM index of 0.9098 was achieved, with 1 being the same as the reference.

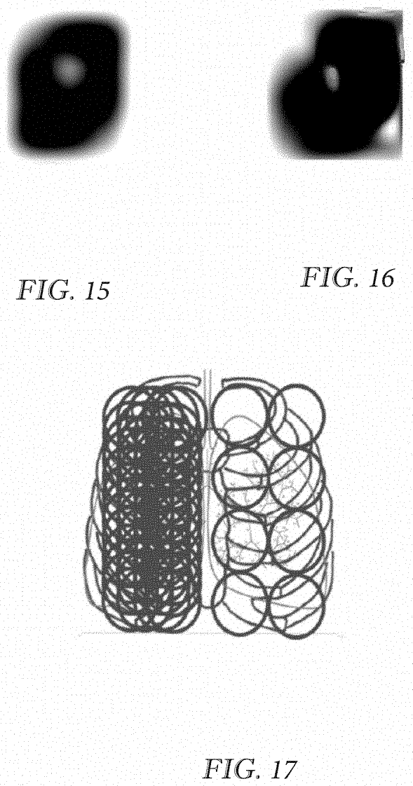

The empirical analysis has demonstrated success in the assessment of lung function, such as presenting regional obstructed airways through acoustic imaging. All 35 different airway segment sizes were included for the impedance and pressure analytical computation, starting with the trachea at n=35 and terminating at the terminal bronchiole with n=1. For the acoustic imaging, only large airways L>2 mm between n=18 and n=35 were utilized in the acoustic simulation of images with the imaging algorithm, as small airways L≤2 mm do not produce an acoustic signal because the flow is laminar and hence silent. A slight difference in the outline and obstructed area in FIGS. 15 and 16 is because the acoustic images were generated from two different sources. The resolution of the lung image was intended mainly for assessing obstructed airways due to the signal resolution. In addition, the airway geometry was drawn ideally so that the airway does not cross, and the bifurcate angles were assumed to be between 45 and 60 degrees.

Model verification through regional pathology has been demonstrated as described above. Additionally, a determination of the relationship between sensor positions, the number of sensors, and nidus localization for HFCWO smart therapy has been established. The effect of sensor distribution on the nidus localization is discussed in more detail below.

A lung size is roughly 260 mm (height) by 110 mm (width). Similar to the actual lung size, the respiratory system modeling disclosed herein was kept within 90% of the actual lung size at about 240 mm (height) by 100 mm (width), as shown in FIG. 17. As the small airway, e.g., diameter less than 2 mm, does not produce an acoustic signal because the flow is laminar and hence silent, the smallest airway diameter simulated in the respiratory system model (see FIG. 9) is about 2 mm as compared to the actual lung, with smallest airway diameter at roughly 0.8 mm. Furthermore, regarding the airway diameters, standard equipment like CT only allows the reconstruction of airway models up to around 2 mm.

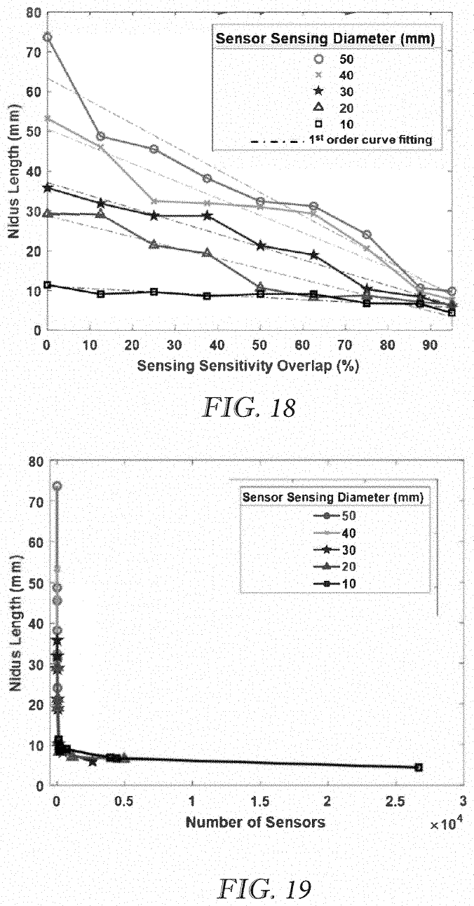

Sensors sensing diameters between 10 mm and 50 mm, with 10 mm increment, and the sensor sensing area overlapping of 0%, 12.5%, 25%, 37.5%, 50%, 62.5%, 75%, 87.5% and 95% were chosen (see FIG. 17) to illustrate the effect of the sensor sensing diameter and the effect of the sensor sensitivity overlap. The sensing diameters were chosen similarly to the commercially available product. The acoustic imaging and airway obstruction were selected in a similar condition doscissed above. The correlation between sensor sensitivity and nidus localization is presented in FIG. 18 and FIG. 19.

The sensor sensing area is denoted as a circle on the chest, with the sensing area overlapping 75% and 0% on the left and right sides of the lung, respectively. The results in FIG. 18 showed that the obstructed airway area's identified diameter with the largest diameter of 72 mm with 50 mm sensing diameter and 0% overlapping. In comparison, the smallest obstructed airway area identified is about 4.2 mm, with a 10 mm sensing diameter and 95% sensing overlap.

In FIG. 19, the number of sensors increased as the sensor sensing area overlapping increased with a sensor sensing diameter of 50 mm with 0% sensor sensitivity overlapping, requiring about 6 sensors. In contrast, about 26,000 sensors are required to achieve a 95% overlapping sensing sensitivity with a 10 mm sensor sensing diameter. The findings in the simulation studies correspond with the understanding that the image resolution increases with the number of sensors. Regardless of the sensing diameter, a similar obstructed nidus length of about 7-9 mm can be identified with a 95% overlapped sensing area as indicated in FIG. 18. This is in line with the comparable number of sensors, at about 90-500 sensors as shown in FIG. 19. A guideline on the number of sensors and specification in the detection of the obstructed area, in terms of nidus length is presented in FIG. 18 and FIG. 19. Furthermore, the practicality in the localization of the nidus length, in terms of the number of sensors required is demonstrated in FIG. 19.

This disclosure demonstrates the respiratory model systems' capacity to pinpoint the source of airway obstruction through acoustic signals, in terms of the nidus length identified to improve HFCWO therapy. The current disclosure relates the location of obstructed airways to both the position and number of acoustic sensors. However, there will be variation in respiratory system model performance due to a range of factors such as the system network architecture: node position in the x- and y-axis location, and the physical airway model: Horsfield or Weibel airway model. The results provided in this disclosure are based on the respiratory model's standalone capabilities for optimizing both the position and quantity of acoustic sensors for gathering usable acoustic information and do not include a different fanciful combination of acoustic sensor's position, e.g., unequal spacing of the placement of the array of sensors. Also, the length of the obstructed airway is determined as the diameter of the obstructed lung area, the perfect circle area identified with the results in FIG. 17-FIG. 19. The airway geometry was assumed to be projected from a 3D to a 2D plane with no intersection between airways, and simulated obstructed area were chosen carefully to avoid outliners in the localization of the nidus length. Lastly, the obstructed area in the simulated lung model can be identified ideally as the model is assumed to be noise-free.

An experimental study on nidus localization was performed to support the findings in the simulation studies. Micro-electromechanical systems (MEMS) microphones were arranged in a 4×6 array to collect lung sound signals. The lung sound signals were collected from the volunteers in an uncontrolled environment consisting of an array of microphones with a sampling frequency of Fs=4000 Hz, which is similar to the literature. A first reference image (healthy) was produced, followed by localization of nidus by positioning different waterbags to imitate the obstructed areas in the airways. The acoustic signals were recorded simultaneously and converted into acoustic images, as discussed in the simulation study. Nidus localizations were successfully demonstrated by the acoustic imaging with the imitation of the obstruction of airways. Thus, the applicability of the simulation study was verified.

Using this approach, the disclosed approach to acoustical lung imaging provides additional information with regard to localization of nidi which can then be used by a caregiver to tailor the therapy applied through HFCWO by varying the intensity by actuator to direct the therapy where it is most needed. This improves the outcomes for the patient and reduces the strain on the patient from an over use of HFCWO.

The present disclosure demonstrates an acoustic intensity imaging tool using one or more sensors 210 (e.g. multi-microphone sensor array 122) to capture an acoustic signal. The current application further provides patients an ‘equipment-to-patient’ approach with the potential for home-based usage, enables frequent monitoring/assessment of lung function for caregivers to adjust the therapy or assess early respiratory disease symptoms, and provides regional information that is a more sensitive tool than the tools that can only provide global assessment/information.

Techniques for assessing local, regional changes in the airways and related to airflow and resistance are useful to caregivers. Frequent outcome measures based on local information may provide vital information to the caregivers. With the information on airway clearance therapy (ACT) effectiveness, caregivers may be able to identify a therapy's suitability and make a timely adjustment to the therapy. Access to sensor technologies and data from respiratory soundwaves' processing has minimized the subjective nature and caregiver's expertise. Imaging can measure lung function regional and help overcome clinical challenges that other outcome measure tools face. Imaging is a sensitive measure to quantify local, regional changes in the lung pathology. Lung sound and vibration energy produced from the chest wall can be transformed into information that presents local ventilation status. Imaging techniques presenting the local, regional changes and ventilation status could increase knowledge regarding ACTs' effectiveness. The disclosed application of multi-sensor imaging has provided new insights into the pathological lung mechanisms by enabling localized assessment.

### APPENDIX

The material parameters of the respiratory system are given in Table A1, and the segment in the k-th layer has the following parameters,

$$\{ \begin{matrix}{Z}_{s} ( k , ω ) \approx ( {R}_{0} ( k ) + j ω {L}_{0} ( k ) ) l ( k ) = \frac{j {ωρ}_{g} l ( k )}{A ( k ) ( 1 - {F}_{v} ( k , ω ) )} \\ {Y}_{s} ( k , ω ) \approx \frac{1}{2} ( {G}_{0} ( k ) + j ω {C}_{0} ( k ) ) l ( k ) \\ = \frac{j ω A ( k ) l ( n )}{2 {ρ}_{g} {v}_{g}^{2}} ( 1 + 0.402 {F}_{t} ( k , ω ) ) + \frac{l ( n )}{2 {Z}_{w} ( k , ω )}\end{matrix} \\ where \{ \begin{matrix}{F}_{v} ( k , ω ) = \frac{2}{{z}_{v}} \frac{{J}_{1} ( {z}_{v} )}{{J}_{0} ( {z}_{v} )} , {z}_{v} = α ( k ) \sqrt{- j ω {ρ}_{g} / {η}_{g}} \\ {F}_{t} ( ω ) = \frac{2}{{z}_{f}} \frac{{J}_{1} ( {z}_{t} )}{{J}_{0} ( {z}_{t} )} , {z}_{t} = α ( k ) \sqrt{- j ω {C}_{g} / {K}_{g}} \\ \frac{1}{{Z}_{w} ( k , ω )} = \frac{c ( k )}{{Z}_{c} ( k , ω )} + \frac{( 1 - c ( k ) )}{{Z}_{s} ( k , ω )}\end{matrix} \\ and \{ \begin{matrix}{Z}_{i} ( k , ω ) = {R}_{i} ( k ) + j ω {I}_{i} ( k ) + \frac{1}{j ω {C}_{i} ( k )} \\ {R}_{i} ( k , ω ) = \frac{4 h ( k ) {E}_{Ii}}{π {d ( k )}^{3} l ( k ) ω} \\ {I}_{i} ( k ) = \frac{h ( k ) {ρ}_{i}}{π d ( k ) l ( k )} \\ {C}_{i} ( k ) = \frac{π {d ( k )}^{3} l ( k )}{4 h ( k ) {E}_{Ri}}\end{matrix} , i = c or s .$$

r_{g}, h_{g}, C_{g}, K_{g} are denoted as air density, viscosity, air specific heat, and thermal conductivity, respectively. Fᵥ and Fₜ account for the sound attenuation by air viscosity and sound attenuation by thermal dissipation, computed with series expansion with J₀ and J₁ being Bessel functions of 0th and 1st order. Z_{w} represents the wall impedance, which is computed from a series of resistance Rᵢ, inductance Iᵢ, and conductance Cᵢ of the acoustic transmission line where rᵢ denotes the density, E_{Ri} and E_{Ii} denote the real and imaginary part of Young's modulus with the subscript i replaced by c for the cartilage or by s for the soft tissue, respectively. The wall thickness, tube diameter, and segment length are denoted by h, d, and l. The impedance at the far end of the airway is,

$${Z}_{s} ( n , ω ) = \frac{{N}_{t}}{j ω {C}_{g} + \frac{1}{{R}_{t} + j ω {I}_{t} + \frac{1}{j ω {C}_{t}}}} ,$$

where Nₜ denotes the total number (≈2.35×10⁶) of terminal bronchiole segments in the Horsfield human airway model. Based on the Dubois six-element airway model, Cg is the alveolar gas compression compliance. Rₜ, Iₜ, and Cₜ are the terminal tissue resistance, inertia, and compliance, respectively.

Although this disclosure refers to specific embodiments, it will be understood by those skilled in the art that various changes in form and detail may be made without departing from the subject matter set forth in the accompanying claims.

1. A method of developing an image of the function of a lung, the method comprising the steps of:
positioning an array of microphones adjacent on a patient, the array of microphones being spaced from one another according to a predetermined pattern;
collecting a signal from each of the microphones;
processing the signal from each of the microphones to establish an intensity for each signal from each microphone at a first time;
applying an interpolation routine to interpolate intensities at a predefined distribution between the microphones at the first time;
normalizing each of the actual and interpolated intensities according to a predetermined schedule;
generating an image of the lung at the first time by displaying the distribution of normalized intensities at the locations associated with each intensity; and
identifying airway blockage by reviewing the displayed distribution of normalized intensities.

2. The method of claim 1 further comprising the steps of
processing the signal from each of the microphones to establish an intensity for each signal from each microphone at a second time;
applying an interpolation routine to interpolate intensities at a predefined distribution between the microphones at the second time;
normalizing each of the actual and interpolated intensities according to a predetermined schedule; and
generating an image of the lung at the second time by displaying the distribution of normalized intensities at the locations associated with each intensity.

3. The method of claim 2, further comprising the steps of:
comparing the image at the second time to the image at the second time to determine a change in lung function in the interval between the first time and the second time.

4. The method of claim 3, further comprising the step of:
applying a noise filter to the normalized intensities prior to generating the image.

5. The method of claim 4, wherein the step of processing the signal includes applying a hybrid approach of signal processing that utilizes wavelet-based total variation and a Wiener filter to limit the signals to frequencies associated with lung function.

6. The method of claim 4, wherein the step of processing the signal includes applying a denoising filter to limit the signals to frequencies associated with lung function.

7. The method of claim 1, wherein the step of processing the signal includes applying a hybrid approach of signal processing that utilizes wavelet-based total variation and a Wiener filter to limit the signals to frequencies associated with lung function.

8. The method of claim 1, further comprising the step of:
applying a noise filter to the normalized intensities prior to generating the image.

9. The method of claim 8, wherein the step of processing the signal includes applying a hybrid approach of signal processing that utilizes wavelet-based total variation and a Wiener filter to limit the signals to frequencies associated with lung function.

10. The method of claim 1, wherein the image at the first time is rendered graphically in gray scale.

11. A garment wearable by and individual, the garment comprising
an array of microphones positioned on the garment in a predetermined pattern, the microphones positioned such when the garment is worn by a person, the microphones are positioned to detects sounds emanating from the lungs and airways of the person,
a computing device positioned on the garment, the computing device including a processor and a memory device, the memory device including instructions that, when executed by the processor, cause the processor to:
collect a signal from each of the microphones;
process the signal from each of the microphones to establish an intensity for each signal from each microphone at a first time;
apply an interpolation routine to interpolate intensities at a predefined distribution between the microphones at the first time;
normalize each of the actual and interpolated intensities according to a predetermined schedule; and
generate an image of the lung at the first time by displaying the distribution of normalized intensities at the locations associated with each intensity.

12. The garment of claim 11, wherein the memory device includes further instructions that, when executed by the processor, cause the processor to:
process the signal from each of the microphones to establish an intensity for each signal from each microphone at a second time;
apply an interpolation routine to interpolate intensities at a predefined distribution between the microphones at the second time;
normalize each of the actual and interpolated intensities according to a predetermined schedule; and
generate an image of the lung at the second time by displaying the distribution of normalized intensities at the locations associated with each intensity.

13. The garment of claim 12, wherein the memory device includes further instructions that, when executed by the processor, cause the processor to:
process the signal by applying a hybrid approach of signal processing that utilizes wavelet-based total variation and a Wiener filter to limit the signals to frequencies associated with lung function.

14. The garment of claim 13, wherein the memory device includes further instructions that, when executed by the processor, cause the processor to:
generate the images of the lung at the first and second time in grayscale, the grayscale based on the intensity of the signal at each location.

15. The garment of claim 14, wherein the memory device includes further instructions that, when executed by the processor, cause the processor to:
apply a noise filter to the normalized intensities prior to generating the images.

16. The garment of claim 15, wherein the memory device includes further instructions that, when executed by the processor, cause the processor to:
apply a denoising filter to limit the signals to frequencies associated with lung function.

17. The garment of claim 12, wherein the memory device includes further instructions that, when executed by the processor, cause the processor to:
generate the images of the lung at the first and second time in grayscale, the grayscale based on the intensity of the signal at each location.

18. The garment of claim 17, wherein the memory device includes further instructions that, when executed by the processor, cause the processor to:
apply a noise filter to the normalized intensities prior to generating the images.

19. The garment of claim 18, wherein the memory device includes further instructions that, when executed by the processor, cause the processor to:
apply a denoising filter to limit the signals to frequencies associated with lung function.

20. The garment of claim 12, wherein the garment further comprises a display supported on the garment, the display in communication with the computer device such that the images generated are displayed on the display on the garment.

21. The garment of claim 20, wherein the microphones are embodied as micro-electro-mechanical system microphones.

22. The garment of claim 21, wherein the microphones are operated passively.
